(19) United States
(12) Patent Application Publication
(10) Pub. No.: US 2006/0193807 A1
(43) Pub. Date: Aug. 31, 2006

(54) Charging composition for coating keratin fibers comprising an aprotic wax

(76) Inventors: Nathalie Jager Lezer, Verrieres-le-Buisson (FR); Frederic Auguste, Chevilly Larue (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/347,252

(22) Filed: Feb. 6, 2006

Related U.S. Application Data
(60) Provisional application No. 60/652,728, filed on Feb. 15, 2005.

(30) Foreign Application Priority Data
Feb. 4, 2005 (FR) 05 50335

(51) Int. Cl. A61K 8/73

(52) U.S. Cl. 424/70.13

(57) ABSTRACT

The present disclosure relates to a composition for coating keratin fibers, comprising, in a cosmetically acceptable continuous aqueous medium, at least one aprotic wax in an amount of greater than 25% by weight relative to the total weight of the composition, said composition having a dry extract of greater than or equal to 40%. Another aspect of the disclosure relates to a composition for coating keratin fibers, comprising, in a cosmetically acceptable continuous aqueous medium, at least 20% of at least one aprotic wax and at least one film-forming polymer chosen from cellulose derivatives. The disclosure also relates to the use of such a composition to obtain charging makeup on keratin fibers and/or a smooth and uniform deposit on the keratin fibers.

This application claims benefit of U.S. Provisional Application No. 60/652,728, filed Feb. 15, 2005, the contents of which are incorporated herein by reference. This application also claims benefit of priority under 35 U.S.C. § 119 to French Patent Application No. 05 50335, filed Feb. 4, 2005, the contents of which are also incorporated herein by reference.

The present disclosure relates to a cosmetic composition for coating keratin fibers.

The composition according to the present disclosure may be a makeup composition, also known as a mascara, a makeup base for keratin fibers, or base coat, a composition to be applied over a makeup, also known as a top coat, or a composition for treating keratin fibers.

In some embodiments, the composition according to the present disclosure is a leave-in composition.

In at least one embodiment, the composition according to the present disclosure is a mascara.

The term “mascara” means a composition intended to be applied to the eyelashes: it may be an eyelash makeup composition, an eyelash makeup base, a composition to be applied over a mascara, also known as a top coat, and a cosmetic eyelash treatment composition. The mascara is intended for human eyelashes, but also for false eyelashes.

Eye makeup compositions, also known as “mascaras”, generally consist of a wax or a mixture of waxes dispersed using at least one surfactant in an aqueous phase also containing polymers and pigments.

It is generally by means of the qualitative and quantitative choice of the waxes and polymers that the desired application specificities for makeup compositions are adjusted, for instance their fluidity, their covering power and/or their curling power. Thus, it is possible to produce various compositions, which, when applied, for example, to the eyelashes, induce a variety of effects such as lengthening, curling and/or thickening (charging effect).

One aspect of the present disclosure is directed towards proposing a composition that is useful for producing a thick makeup result on keratin fibers, for example on the eyelashes, which is also known as charging makeup. According to the present disclosure, the term “keratin fibers” covers the hair, the eyelashes and the eyebrows and also extends to synthetic wigs and false eyelashes.

It is known from the prior art that the higher the solids content (provided in part by a fatty phase consisting, for example, of one or more waxes or of one or more lipophilic polymers) in a composition, the greater will be the amount of material deposited on the eyelash, and thus the more volumizing will be the result obtained.

However, increasing the solids content in a composition, such as an emulsion or dispersion, may result in an increase in the consistency of the product obtained and thus may require intricate and difficult application to the eyelashes since the product may be thick, viscous, difficult to deposit, deposit non-uniformly and in lumps, and the makeup thus obtained may have a coarse, granular appearance; the resulting makeup may not be uniform and may look unattractive.

Another means for increasing the solids content is to incorporate solid particles, for example with the addition of fillers or pigments. However, the increase in consistency also limits the maximum percentage of solids. Further, the use of solid particles in large amounts does not necessarily result in a smooth and uniform deposit, not only because of the consistency but also because of the size of the particles introduced, thus possiblygiving the deposit an overall granular, coarse appearance.

This is generally the case for “volumizing” mascaras, which may be difficult to apply and may give a non-uniform deposit.

It is thus difficult to obtain a keratin fiber makeup composition with a high solids content and thus a satisfactory volumizing effect that is easy to apply and/or that gives a uniform deposit.

Unexpectedly, it has been found by the present inventors that it is possible to prepare compositions with a high dry extract, which can give a thickening makeup result on keratin fibers and a smooth and uniform deposit on the said fibers, by means of using in these compositions an aprotic wax in a certain content.

For the purposes of the present disclosure, the term “charging” means thick and volumizing makeup on keratin fibers, such as the eyelashes.

In at least one embodiment, the compositions according to the present disclosure have a solids content of greater than or equal to 40% by weight, such as greater than or equal to 42% by weight, and further for example greater than or equal to 45% by weight and even further for example greater than or equal to 47% by weight, which may be up to 70% by weight, relative to the total weight of the composition.

The solids content, i.e., the content of non-volatile matter, may be measured in different ways: mention may be made, for example, of the methods of oven drying, the methods of drying by exposure to infrared radiation and also the chemical methods of Karl Fischer water titration.

In at least one embodiment, the amount of solids, commonly referred to as the “dry extract”, of the compositions according to the present disclosure is measured by heating the sample with infrared rays with a wavelength of from 2 μm to 3.5 μm. The substances contained in the compositions that have a high vapor pressure evaporate under the effect of this radiation. Measurement of the weight loss of the sample makes it possible to determine the “dry extract” of the composition. These measurements are performed using an LP 16 commercial infrared desiccator from Mettler. This technique is fully described in the machine documentation supplied by Mettler.

The measuring protocol is as follows:

About 1 g of the composition is spread out on a metal crucible. This crucible, after being placed in the desiccator, is subjected to a nominal temperature of 120° C. for one hour. The wet mass of the sample, corresponding to the initial mass, and the dry mass of the sample, corresponding to the mass after exposure to the radiation, are measured using a precision balance.

The solids content is calculated in the following manner:
Dry extract=100×(dry mass/wet mass).

According to one of its aspects, a subject of the present disclosure is thus a composition for coating keratin fibers, comprising a cosmetically acceptable continuous aqueous medium and at least one aprotic wax in an amount of greater than 25% by weight relative to the total weight of the composition, the composition having a dry extract of greater than or equal to 40%.

According to another aspect, a subject of the disclosure is also a composition for coating keratin fibers, comprising a cosmetically acceptable continuous aqueous medium, characterized in that it comprises at least 20% of at least one aprotic wax and at least one film-forming polymer chosen from cellulose derivatives.

The term “composition with a “continuous” aqueous medium,” as used herein, means that the composition has a conductivity, measured at 25° C., of greater than or equal to 23 μS/cm (microSiemens/cm), the conductivity being measured, for example, using an MPC227 conductimeter from Mettler Toledo and an Inlab 730 conductivity measuring cell. The measuring cell is immersed in the composition, so as to remove the air bubbles liable to form between the two electrodes of the cell. The conductivity reading is taken when the conductimeter value has stabilized. A mean is calculated on at least three successive measurements.

The present disclosure is also directed towards a process for making up keratin fibers, wherein a composition in accordance with the disclosure is applied to the fibers.

The present disclosure also relates to the use of a composition in order to obtain charging makeup on keratin fibers, for example on the eyelashes and the eyebrows and/or a smooth and uniform deposit on the said fibers.

Another aspect of the present disclosure is also the use, in a composition comprising a cosmetically acceptable continuous aqueous medium, of at least one aprotic wax to obtain charging makeup on keratin fibers, for example on the eyelashes and the eyebrows and a smooth and uniform deposit on the said fibers.

Aprotic Wax

In the context of the present disclosure, a wax may be defined as being a lipophilic compound that is solid at room temperature (25° C.), with a solid/liquid reversible change of state, having a melting point of greater than or equal to 30° C., which may be up to 120° C.

By bringing the wax to the liquid form (melting), it is possible to make it miscible with oils and to form a microscopically uniform mixture, but on cooling the mixture to room temperature, recrystallization of the wax in the oils of the mixture is obtained.

In one embodiment, for example, the waxes that are suitable for use herein may have a melting point of greater than 45° C. approximately, and further greater than or equal to 50° C. and and even further greater than or equal to 55° C.

The melting point of the wax may be measured using a differential scanning calorimeter (DSC), for example the calorimeter sold under the name MDSC 2929 by the company TA Instruments.

The measuring protocol is as follows:

A sample of 5 mg of product placed in a crucible is subjected to a first temperature rise ranging from 0° C. to 120° C., at a heating rate of 10° C./minute, it is then cooled from 120° C. to 0° C. at a cooling rate of 10° C./minute and is finally subjected to a second temperature increase ranging from 0° C. to 120° C. at a heating rate of 5° C./minute. During the second temperature increase, the variation of the difference in power absorbed by the empty crucible and by the crucible containing the sample of product is measured as a function of the temperature. The melting point of the compound is the temperature value corresponding to the top of the peak of the curve representing the variation in the difference in absorbed power as a function of the temperature.

As used herein, the term “aprotic wax” means a wax comprising few or no hydrogen atoms linked to a highly electronegative atom such as O or N.

The aprotic waxes may be chosen from apolar waxes, in other words waxes consisting solely of molecules comprising only carbon and hydrogen atoms in their chemical structure, and in other words comprising no hetero atoms (such as O, N or P).

Examples of aprotic waxes, for instance apolar waxes, which may be mentioned include but are not limited to paraffin wax, microcrystalline waxes, ozokerite, ceresin and synthetic waxes, for instance polymethylene wax, polyethylene wax, propylene wax and ethylene/propylene copolymers thereof, and Fischer-Tropsch waxes, and mixtures thereof.

The waxes obtained by esterification or modified by esterification and which may comprise residual OH groups as a function of the esterification yield may be considered as aprotic within the meaning of the present disclosure. Such waxes are, for example, the wax obtained from the reaction of a fatty acid with a branched polyol of bis(trimethylol) type, for instance those sold under the name HEST by the company Heterene. Mention may also be made of silicone-modified waxes, for instance the silicone-treated candelilla wax sold by Koster Keunen under the name SILICONYL CANDELILLA.

The waxes obtained by catalytic hydrogenation of animal or plant oils containing linear or branched C8-C32 fatty chains, such as hydrogenated jojoba oil, hydrogenated sunflower oil or hydrogenated coconut oil, or the wax obtained by hydrogenation of olive oil esterified with stearyl alcohol, may also be considered as aprotic.

Conversely, waxes that are considered as protic are hydrocarbon-based waxes, for instance beeswax or lanolin wax; orange wax, lemon wax, rice bran wax, carnauba wax, candelilla wax, ouricurry wax, Japan wax, berry wax, shellac wax and sumac wax; montan wax, hydrogenated castor oil, hydrogenated lanolin oil, the waxes obtained from the reaction of fatty acids with carbohydrates, for instance disaccharides of sucrose type, such as sucrose polybehenate, sold by Croda under the name CROMADERM B, and hydroxy ester waxes, for instance C₂₀-C₄₀ alkyl (hydroxystearyloxy)stearate wax, such as those sold under the names “Kester Wax K 82 P®” and “Kester Wax K 80 P®” by the company Koster Keunen.

For example, synthetic aprotic waxes are suitable for use, such as paraffin waxes, ceresin wax and ozokerite wax, polymethylene waxes, including the waxes Cirebelle 303® and Cirebelle 505® sold by the company Sasol, polyethylene wax, propylene wax and ethylene/propylene copolymers thereof, and Fischer-Tropsch waxes, and mixtures thereof.

Mention may also be made of polyolefin waxes such as those derived from the polymerization, e.g., the homopolymerization, of an α-olefin corresponding to the general formula: R—CH═CH₂ in which R denotes an alkyl radical, which may be a linear alkyl radical, containing from 10 to 50 carbon atoms such as from 25 to 50 carbon atoms.

The term “α-olefin homopolymerization,” as used herein, means the polymerization of monomers consisting essentially of an α-olefin or a mixture of α-olefins.

Such polyolefin waxes may have a number-average molecular weight ranging from 400 to 3000, such as from 2000 to 3000 and, for example, such as from 2500 to 2700.

Such polyolefin waxes are described in U.S. Pat. No. 4,060,569 and U.S. Pat. No. 4,239,546. These waxes include those sold under the name “Performa V® 103”, “Performa V® 253” and “Performa V® 260” by the company Petrolite.

According to at least one embodiment, for example, the aprotic wax(es) may be present in an amount ranging from 20% to 60% by weight, and further from 25% to 60% by weight, such as from 27% to 50% by weight and further for example from 28% to 45% by weight relative to the total weight of the composition.

According to another aspect, the composition according to the present disclosure comprises less than 5% by weight, and further less than 4%, such as less than 3% and even further for example less than 2% by weight of protic wax(es), as defined above, relative to the total weight of the composition.

Accordingly, another subject of the disclosure is a composition for coating keratin fibers, comprising a cosmetically acceptable continuous aqueous medium, which comprises less than 5% by weight of protic wax relative to the total weight of the composition.

In some embodiments, the composition according to the present disclosure is free of protic wax.

Aqueous Medium

The cosmetically acceptable continuous aqueous medium of the composition, as disclosed herein, may consist essentially of water; it may also comprise a mixture of water and of water-miscible solvent (water miscibility of greater than 50% by weight at 25° C.), for instance lower monoalcohols containing from 1 to 5 carbon atoms, such as ethanol and isopropanol, glycols containing from 2 to 8 carbon atoms, such as propylene glycol, ethylene glycol, 1,3-butylene glycol and dipropylene glycol, C₃-C₄ ketones and C₂-C₄ aldehydes, and mixtures thereof.

The aqueous medium (water and, optionally, the water-miscible solvent) may be present in a content ranging from 0.1% to 95% by weight and further ranging from 1% to 80% by weight relative to the total weight of the composition.

Emulsifying System

The composition according to the present disclosure may contain emulsifying surfactants present, for example in an amount ranging from 0.1% to 30%, and further from 1% to 15% and such as from 2% to 10% by weight relative to the total weight of the composition.

According to the present disclosure, an emulsifier appropriately chosen to obtain an oil-in-water emulsion is generally used. For instance, an emulsifier having at 25° C. an HLB (hydrophilic-lipophilic balance), in the Griffin sense, of greater than or equal to 8 may be used.

The HLB value according to Griffin is defined in J. Soc. Cosm. Chem. 1954 (volume 5), pages 249-256.

These surfactants may be chosen from nonionic, anionic, cationic and amphoteric surfactants, and emulsifying surfactants. Reference may be made to the document “Encyclopedia of Chemical Technology, Kirk-Othmer”, volume 22, pp. 333-432, 3rd edition, 1979, Wiley, for the definition of the properties and (emulsifying) functions of surfactants, specifically pp. 347-377 of this reference, for anionic, amphoteric and nonionic surfactants.

In at least one embodiment, the surfactants used in the composition according to the present disclosure are chosen from:

a) nonionic surfactants with an HLB of greater than or equal to 8 at 25° C., used alone or as a mixture; for example, mention may be made of:

oxyethylenated and/or oxypropylenated ethers (which may comprise from 1 to 150 oxyethylene and/or oxypropylene groups) of glycerol;

oxyethylenated and/or oxypropylenated ethers (which may comprise from 1 to 150 oxyethylene and/or oxypropylene groups) of fatty alcohols (for instance C8-C24, e.g., C12-C18, alcohol), such as oxyethylenated cetearyl alcohol ether containing 30 oxyethylene groups (CTFA name “Ceteareth-30”) and the oxyethylenated ether of the mixture of C12-C15 fatty alcohols comprising 7 oxyethylene groups (CTFA name “C12-15 Pareth-7” sold under the name Neodol 25-7® by Shell Chemicals);

fatty acid esters (for instance a C8-C24, such as a C16-C22 acid) of polyethylene glycol (which may comprise from 1 to 150 ethylene glycol units), such as PEG-50 stearate and PEG-40 monostearate sold under the name Myrj 52P® by the company ICI Uniqema;

fatty acid esters (for instance a C8-C24 such as a C16-C22 acid) of oxyethylenated and/or oxypropylenated glyceryl ethers (which may comprise from 1 to 150 oxyethylene and/or oxypropylene groups), for instance PEG-200 glyceryl monostearate sold under the name Simulsol 220™® by the company SEPPIC; glyceryl stearate polyethoxylated with 30 ethylene oxide groups, for instance the product Tagat S® sold by the company Goldschmidt, glyceryl oleate polyethoxylated with 30 ethylene oxide groups, for instance the product Tagat O® sold by the company Goldschmidt, glyceryl cocoate polyethoxylated with 30 ethylene oxide groups, for instance the product Varionic LI 13® sold by the company Sherex, glyceryl isostearate polyethoxylated with 30 ethylene oxide groups, for instance the product Tagat L® sold by the company Goldschmidt, and glyceryl laurate polyethoxylated with 30 ethylene oxide groups, for instance the product Tagat I® from the company Goldschmidt;

fatty acid esters (for instance a C8-C24 such as aa C16-C22 acid) of oxyethylenated and/or oxypropylenated sorbitol ethers (which may comprise from 1 to 150 oxyethylene and/or oxypropylene groups), for instance polysorbate 60 sold under the name Tween 60® by the company Uniqema;

dimethicone copolyol, such as the product sold under the name Q2-5220® by the company Dow Corning;

dimethicone copolyol benzoate (Finsolv SLB 101® and 201® by the company Finetex);

copolymers of propylene oxide and of ethylene oxide, also known as EO/PO polycondensates;

and mixtures thereof.

The EO/PO polycondensates used herein are, for example, copolymers comprising polyethylene glycol and polypropylene glycol blocks, for instance polyethylene glycol/polypropylene glycol/polyethylene glycol triblock polycondensates. These triblock polycondensates have, for example, the following chemical structure:
H—(O—CH₂—CH₂)ₐ—(O—CH(CH₃)—CH₂)_{b}—(O—CH₂—CH₂)ₐ—OH,

in which formula a ranges from 2 to 120 and b ranges from 1 to 100.

In at least one embodiment, the EO/PO polycondensate has a weight-average molecular weight ranging from 1000 to 15 000 and such as ranging from 2000 to 13 000. The said EO/PO polycondensate has a cloud point, at 10 g/l in distilled water, of greater than or equal to 20° C., such as greater than or equal to 60° C. The cloud point is measured according to ISO standard 1065. As EO/PO polycondensates that may be used according to the present disclosure, mention may be made of the polyethylene glycol/polypropylene glycol/polyethylene glycol triblock polycondensates sold under the name Synperonic®, for instance Synperonic PE/L44® and Synperonic PE/F127®, by the company ICI.

b) nonionic surfactants with an HLB of less than 8 at 25° C., optionally combined with one or more nonionic surfactants with an HLB of greater than 8 at 25° C., such as those mentioned above, such as:

saccharide esters and ethers, such as sucrose stearate, sucrose cocoate and sorbitan stearate, and mixtures thereof, for instance Arlatone 2121® sold by the company ICI;

fatty acid esters (for instance of a C8-C24, such as of a C16-C22, acid) of polyols, e.g., of glycerol or sorbitol, such as glyceryl stearate, glyceryl stearate such as the product sold under the name Tegin M® by the company Goldschmidt, glyceryl laurate such as the product sold under the name Imwitor 312® by the company Hüls, polyglyceryl-2 stearate, sorbitan tristearate or glyceryl ricinoleate;

the mixture of cyclomethicone/dimethicone copolyol sold under the name of Q2-3225C® by the company Dow Corning.

c) anionic surfactants such as:

C16-C30 fatty acid salts, for example those derived from amines, for instance triethanolamine stearate and/or 2-amino-2-methyl-1,3-propanediol stearate;

polyoxyethylenated fatty acid salts, for example those derived from amines or alkali metal salts, and mixtures thereof;

phosphoric esters and salts thereof, such as “DEA oleth-10 phosphate” (CRODAFOS N 10N from the company Croda) or monocetyl monopotassium phosphate (AMPHISOL K from Givaudan);

sulfosuccinates such as “Disodium PEG-5 citrate lauryl sulfosuccinate” and “Disodium ricinoleamido MEA sulfosuccinate”;

alkyl ether sulfates, such as sodium lauryl ether sulfate;

isethionates;

acylglutamates such as “Disodium hydrogenated tallow glutamate” (Amisoft HS-21 R® sold by the company Ajinomoto), and mixtures thereof.

According to at least one embodiment, triethanolamine stearate and/or 2-amino-2-methyl-1,3-propanediol are useful as surfactants herein. These surfactants are generally obtained by simple mixing of stearic acid and triethanolamine and/or of 2-amino-2-methyl-1,3-propanediol.

The compositions according to the present disclosure may also contain one or more amphoteric surfactants, for instance N-acylamino acids such as N-alkylaminoacetates and disodium cocoamphodiacetate, and amine oxides such as stearamine oxide, and/or alternatively silicone surfactants, for instance dimethicone copolyol phosphates such as the product sold under the name Pecosil PS 100® by the company Phoenix Chemical.

Film-Forming Polymer

The composition as disclosed herein may comprise a film-forming polymer. As used herein, the term “film-forming polymer” means a polymer capable, by itself or in the presence of an auxiliary film-forming agent, of forming a continuous film that adheres to a support, for example to keratin materials, which may include a cohesive film and even further a film whose cohesion and mechanical properties are such that the film may be isolated from the support.

The film-forming polymer may be present in the composition according to the present disclosure in a solids content ranging from 0.1% to 60% by weight, such as from 0.5% to 40% by weight and further for example ranging from 1% to 30% by weight relative to the total weight of the composition.

Among the film-forming polymers that may be used in the composition as disclosed herein, mention may be made of synthetic polymers, of free-radical type or of polycondensate type, and polymers of natural origin, and mixtures thereof.

As used herein, the expression “free-radical film-forming polymer” means a polymer obtained by polymerization of unsaturated monomers, for example, ethylenically unsaturated monomers, each monomer being capable of homopolymerizing (unlike polycondensates).

The film-forming polymers of free-radical type may be, for example, vinyl polymers or copolymers, and for example acrylic polymers.

The vinyl film-forming polymers can result from the polymerization of monomers containing ethylenic unsaturation and containing at least one acidic group and/or esters of these acidic monomers and/or amides of these acidic monomers.

Monomers bearing an acidic group that may be used are α,β-ethylenic unsaturated carboxylic acids such as acrylic acid, methacrylic acid, crotonic acid, maleic acid or itaconic acid. (Meth)acrylic acid and crotonic acid may be used, and in at least one embodiment, (meth)acrylic acid is used.

The esters of acidic monomers may be chosen from (meth)acrylic acid esters (also known as (meth)acrylates), for example (meth)acrylates of an alkyl, e.g., a C₁-C₃₀ and further a C₁-C₂₀ alkyl; (meth)acrylates of an aryl, for example a C₆-C₁₀ aryl, and (meth)acrylates of a hydroxyalkyl, such as a C₂-C₆ hydroxyalkyl.

Among the alkyl (meth)acrylates that may be mentioned are methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl (meth)acrylates that may be mentioned are hydroxyethyl acrylate, 2-hydroxypropyl acrylate, hydroxyethyl methacrylate and 2-hydroxy-propyl methacrylate.

Among the aryl (meth)acrylates that may be mentioned are benzyl acrylate and phenyl acrylate.

In at least one embodiment, for example, the (meth)acrylic acid esters are alkyl (meth)acrylates.

According to the present disclosure, the alkyl group of the esters may be either fluorinated or perfluorinated, in other words, some or all of the hydrogen atoms of the alkyl group are substituted with fluorine atoms.

Examples of amides of the acid monomers that may be mentioned are (meth)acrylamides, such as N-alkyl(meth)acrylamides, wherein in at least one embodiment, the alkyl is a C₂-C₁₂ alkyl. Among the N-alkyl(meth)acrylamides that may be mentioned are N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. For instance, these monomers may be polymerized with acid monomers and/or esters thereof and/or amides thereof, such as those mentioned above.

Examples of vinyl esters that may be mentioned are vinyl acetate, vinyl neodecanoate, vinyl pivalate, vinyl benzoate and vinyl t-butylbenzoate. Styrene monomers may include, but are not limited to styrene and α-methylstyrene.

The film-forming polycondensates that may be mentioned may include, but are not limited to polyurethanes, polyesters, polyesteramides, polyamides, epoxyester resins and polyureas.

The polyurethanes may be chosen from anionic, cationic, nonionic and amphoteric polyurethanes, polyurethane-acrylics, polyurethane-polyvinylpyrrolidones, polyester-polyurethanes, polyether-polyurethanes, polyureas and polyurea/polyurethanes, and mixtures thereof.

The polyesters may be obtained, in a known manner, by polycondensation of dicarboxylic acids with polyols, for example diols.

The dicarboxylic acid may be aliphatic, alicyclic or aromatic. Examples of such acids that may be mentioned are: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid or 2,6-naphthalenedicarboxylic acid. These dicarboxylic acid monomers may be used alone or as a combination of at least two dicarboxylic acid monomers. For example, the monomers used may be phthalic acid, isophthalic acid and/or terephthalic acid.

The diol may be chosen from aliphatic, alicyclic and aromatic diols. In at least one embodiment, the diol used is chosen from: ethylene glycol, propylene glycol, diethylene glycol, triethylene glycol, 1,3-propanediol, cyclohexanedimethanol and 4-butanediol. Other polyols that may be used are glycerol, pentaerythritol, sorbitol and trimethylolpropane.

The polyesteramides may be obtained in a manner analogous to that of the polyesters, by polycondensation of diacids with diamines or amino alcohols. Diamines that may be used are ethylenediamine, hexamethylenediamine and meta- or para-phenylenediamine. An amino alcohol that may be used is monoethanolamine.

The polyester may also comprise at least one monomer bearing at least one group —SO₃M, with M representing a hydrogen atom, an ammonium ion NH₄⁺ or a metal ion such as, for example, an Na⁺, Li⁺, K⁺, Mg²⁺, Ca²⁺, Cu²⁺, Fe²⁺ or Fe³⁺ ion. According to at least one embodiment, for example, a difunctional aromatic monomer comprising such a group —SO₃M may be used.

The aromatic nucleus of the difunctional aromatic monomer also bearing a group —SO₃M as described above may be chosen, for example, from benzene, naphthalene, anthracene, biphenyl, oxybiphenyl, sulfonylbiphenyl and methylenebiphenyl nuclei. As examples of difunctional aromatic monomers also bearing a group —SO₃M, mention may be made of: sulfoisophthalic acid, sulfoterephthalic acid, sulfophthalic acid, 4-sulfonaphthalene-2,7-dicarboxylic acid.

The copolymers that may be used are those based on isophthalate/sulfoisophthalate, for example copolymers obtained by condensation of diethylene glycol, cyclohexanedimethanol, isophthalic acid and sulfoisophthalic acid.

The composition may comprise a water-soluble film-forming polymer chosen from:

proteins, for instance proteins of plant origin such as wheat or soybean proteins; proteins of animal origin such as keratins, for example keratin hydrolysates and sulfonic keratins;

anionic, cationic, amphoteric or nonionic chitin or chitosan polymers;

cellulose polymers such as hydroxyethylcellulose, hydroxypropylcellulose, methylcellulose, ethylhydroxyethylcellulose and carboxymethylcellulose, and also quaternized cellulose derivatives;

vinyl polymers, for instance polyvinylpyrrolidones, copolymers of methyl vinyl ether and of maleic anhydride, the copolymer of vinyl acetate and of crotonic acid, copolymers of vinylpyrrolidone and of vinyl acetate; copolymers of vinylpyrrolidone and of caprolactam; polyvinyl alcohol;

polymers of natural origin, optionally modified, such as:

gum arabics, guar gum, xanthan derivatives and karaya gum;

alginates and carrageenans;

glycoaminoglycans, and hyaluronic acid and its derivatives;

shellac resin, sandarac gum, dammar resins, elemi gums and copal resins;

deoxyribonucleic acid;

mucopolysaccharides such as hyaluronic acid and chondroitin sulfate, and mixtures thereof.

According to at least one embodiment, the composition comprises at least one film-forming polymer chosen from cellulose-based polymers such as alkylcelluloses such as methylcellulose or ethylcellulose, hydroxyalkylcelluloses such as hydroxyethylcellulose, hydroxypropylcellulose or ethylhydroxyethylcellulose, and carboxyalkylcelluloses such as carboxymethylcellulose.

According to another embodiment of the disclosure, the composition comprises at least one film-forming polymer chosen from cellulose derivatives and an aprotic wax present in a content ranging from at least 20% by weight relative to the total weight of the composition.

Accordingly, another aspect of the disclosure is a composition for coating keratin fibers, comprising a cosmetically acceptable continuous aqueous medium, wherein comprises at least one aprotic wax in an amount of greater than or equal to 20% by weight relative to the total weight of the composition and at least one film-forming polymer chosen from cellulose-based polymers.

The film-forming polymer may also be present in the composition in the form of particles dispersed in an aqueous phase or in a non-aqueous solvent phase (liquid organic medium of the composition), which is generally known as a latex or pseudolatex. The techniques for preparing these dispersions are well known to those skilled in the art.

Aqueous dispersions of film-forming polymers that may be used are the acrylic dispersions sold under the names “Neocryl XK-90®”, “Neocryl A-1070®”, “Neocryl A-1090®”, “Neocryl BT-62®”, “Neocryl A-1079®” and “Neocryl A-523®” by the company Avecia-Neoresins, “Dow Latex 432®” by the company Dow Chemical, “Daitosol 5000 AD®” or “Daitosol 5000 SJ®” by the company Daito Kasey Kogyo; “Syntran 5760” by the company Interpolymer or the aqueous dispersions of polyurethane sold under the names “Neorez R-981®” and “Neorez R-974®” by the company Avecia-Neoresins, “Avalure UR-405®”, “Avalure UR-410®”, “Avalure UR-425®”, “Avalure UR-450®”, “Sancure 875®”, “Sancure 861®”, “Sancure 878®” and “Sancure 2060®” by the company Goodrich, “Impranil 85®” by the company Bayer and “Aquamere H-1511®” by the company Hydromer; the sulfopolyesters sold under the brand name “Eastman AQ®” by the company Eastman Chemical Products, and vinyl dispersions, for instance “Mexomer PAM” by the company Chimex, and mixtures thereof.

The composition according to the present disclosure may comprise a plasticizer that promotes the formation of a film with the film-forming polymer. Such a plasticizer may be chosen from any compound known to those skilled in the art as being capable of satisfying the desired function.

The claimed compositions may also contain ingredients commonly used in the field of keratin fiber makeup.

The composition according to the disclosure may comprise one or more oils, which are preferably aprotic.

The oil may be present in the composition according to the disclosure in an amount ranging from 0.1% to 60% by weight, such as from 0.1% to 30% by weight, relative to the total weight of the composition.

The oil may be chosen from volatile oils and non-volatile oils, and mixtures thereof. In at least one embodiment, the composition may comprise at least one volatile oil.

For the purposes of the present disclosure, the term “volatile oil” means an oil that is capable of evaporating on contact with the skin or the keratin fiber in less than one hour, at room temperature and atmospheric pressure. The volatile organic solvent(s) and volatile oils of the present disclosure are volatile organic solvents and cosmetic oils that are liquid at room temperature, with a non-zero vapor pressure at room temperature and atmospheric pressure, ranging from 0.13 Pa to 40 000 Pa (10⁻³ to 300 mmHg), such as from 1.3 Pa to 13 000 Pa (0.01 to 100 mmHg), and even further for example ranging from 1.3 Pa to 1300 Pa (0.01 to 10 mmHg).

As used herein, the term “non-volatile oil” means an oil that remains on the skin or the keratin fiber at room temperature and atmospheric pressure for at least several hours and that, in at least one embodiment, has a vapor pressure of less than 10⁻³ mmHg (0.13 Pa).

These oils may be hydrocarbon-based oils, silicone oils and fluoro oils, and mixtures thereof.

The term “hydrocarbon-based oil,” as used herein, means an oil mainly containing hydrogen and carbon atoms and optionally oxygen, nitrogen, sulfur and phosphorus atoms. The volatile hydrocarbon-based oils may be chosen from hydrocarbon-based oils containing from 8 to 16 carbon atoms, and including branched C8-C16 alkanes, for instance C8-C16 isoalkanes of petroleum origin (also known as isoparaffins), for instance isododecane (also known as 2,2,4,4,6-pentamethylheptane), isodecane and isohexadecane, for example the oils sold under the trade names Isopar or Permethyl, branched C8-C16 esters and isohexyl neopentanoate, and mixtures thereof. Other volatile hydrocarbon-based oils, for instance petroleum distillates, including those sold under the name Shell Solt by the company Shell, may also be used. For example, the volatile solvent may be chosen from volatile hydrocarbon-based oils containing from 8 to 16 carbon atoms, and mixtures thereof.

Volatile oils that may also be used include volatile silicones, for instance volatile linear or cyclic silicone oils, and further those with a viscosity ≦8 centistokes (8×10⁻⁶ m²/s) and containing from 2 to 7 silicon atoms, these silicones optionally comprising alkyl or alkoxy groups containing from 1 to 10 carbon atoms. As volatile silicone oils that may be used in the present disclosure, mention may be made of octamethyl cyclotetrasiloxane, decamethyl cyclopentasiloxane, dodecamethyl cyclo-hexasiloxane, heptamethyl hexyltrisiloxane, heptamethyloctyl trisiloxane, hexamethyl disiloxane, octamethyl trisiloxane, decamethyl tetrasiloxane and dodecamethyl pentasiloxane, and mixtures thereof.

Mention may also be made of the linear volatile alkyltrisiloxane oils of general formula (I): 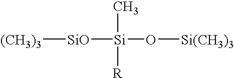
in which R represents an alkyl group containing from 2 to 4 carbon atoms and of which one or more hydrogen atoms may be substituted with one or more fluorine or chlorine atoms.

Among the oils of general formula (I) that may be mentioned are:
3-butyl-1,1,1,3,5,5,5-heptamethyltrisiloxane,
3-propyl-1,1,1,3,5,5,5-heptamethyltrisiloxane, and
3-ethyl-1,1,1,3,5,5,5-heptamethyltrisiloxane,
   corresponding to the oils of formula (I) for which R is, respectively, a butyl group, a propyl group or an ethyl group.

Volatile fluorinated solvents such as nonafluoromethoxybutane and perfluoromethylcyclopentane may also be used.

The composition may also comprise at least one non-volatile oil, which may be chosen from non-volatile hydrocarbon-based oils and silicone oils and fluoro oils.

Non-volatile hydrocarbon-based oils that may be mentioned include:

hydrocarbon-based oils of plant origin, such as triesters of fatty acids and of glycerol, the fatty acids of which may have varied chain lengths from C4 to C24, these chains possibly being linear or branched, and saturated or unsaturated; these oils include wheatgerm oil, sunflower oil, grapeseed oil, sesame seed oil, corn oil, apricot oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, sesame seed oil, marrow oil, rapeseed oil, blackcurrant oil, evening primrose oil, millet oil, barley oil, quinoa oil, rye oil, safflower oil, candlenut oil, passionflower oil and musk rose oil; and caprylic/capric acid triglycerides, for instance those sold by the company Stearineries Dubois and those sold under the names Miglyol 810, 812 and 818 by the company Dynamit Nobel,

synthetic ethers containing from 10 to 40 carbon atoms,

linear or branched hydrocarbons of mineral or synthetic origin, such as petroleum jelly, polydecenes, hydrogenated polyisobutene such as parleam, and squalane, and mixtures thereof,

synthetic esters, for instance oils of formula R1 COOR2 in which R1 represents a linear or branched fatty acid residue containing from 1 to 40 carbon atoms and R2 represents a hydrocarbon-based chain, which may be branched, containing from 1 to 40 carbon atoms, on condition that R1+R2≧10, for instance purcellin oil (cetostearyl octanoate), isopropyl myristate, isopropyl palmitate, C12 to C15 alkyl benzoates, hexyl laurate, diisopropyl adipate, isononyl isononanoate, 2-ethylhexyl palmitate, isostearyl isostearate, octanoates; hydroxylated esters, for instance isostearyl lactate or diisostearyl malate; and pentaerythritol esters,

carbonates,

acetals,

citrates,

and mixtures thereof.

The non-volatile silicone oils that may be used in the composition according to the present disclosure may be non-volatile polydimethylsiloxanes (PDMS), polydimethylsiloxanes comprising alkyl or alkoxy groups, which are pendent and/or at the end of a silicone chain, these groups each contain from 2 to 24 carbon atoms, phenyl silicones, for instance phenyl trimethicones, phenyl dimethicones, phenyltrimethyl-siloxydiphenylsiloxanes, diphenyl dimethicones, diphenylmethyldiphenyltrisiloxanes and 2-phenylethyltrimethylsiloxysilicates.

The fluoro oils that may be used herein include but are not limited to fluorosilicone oils, fluoro polyethers and fluorosilicones as described in published European Patent Application No. EP-A-847 752.

Additives

The composition according to the present disclosure may also comprise a dyestuff, for instance pulverulent dyestuffs, liposoluble dyes and water-soluble dyes.

This dyestuff may be present in an amount ranging from 0.01% to 30% by weight relative to the total weight of the composition.

The pulverulent dyestuffs may be chosen from pigments and nacres.

The pigments may be white or colored, mineral and/or organic, and coated or uncoated. Among the mineral pigments that may be mentioned are titanium dioxide, optionally surface-treated, zirconium oxide, zinc oxide or cerium oxide, and also iron oxide or chromium oxide, manganese violet, ultramarine blue, chromium hydrate and ferric blue. Among the organic pigments that may be mentioned are carbon black, pigments of D & C type, and lakes based on cochineal carmine or on barium, strontium, calcium or aluminium.

The nacres may be chosen from white nacreous pigments such as mica coated with titanium or with bismuth oxychloride, colored nacreous pigments such as titanium mica with iron oxides, titanium mica with, for example, ferric blue and chromium oxide, titanium mica with an organic pigment of the abovementioned type, and also nacreous pigments based on bismuth oxychloride.

The liposoluble dyes are, for example, Sudan Red, D&C Red 17, D&C Green 6,β-carotene, soybean oil, Sudan Brown, D&C Yellow 11, D&C Violet 2, D&C Orange 5, quinoline yellow and annatto. The water-soluble dyes are, for example, beetroot juice, methylene blue, the disodium salt of ponceau, the disodium salt of alizarin green, quinoline yellow, the trisodium salt of amaranth, the disodium salt of tartrazine, the monosodium salt of rhodamine, the disodium salt of fuchsin, and xanthophyll.

The composition according to the present disclosure may also comprise a filler chosen from those that are well known to persons skilled in the art and commonly used in cosmetic compositions. The fillers may be mineral or organic, and lamellar or spherical. Mention may be made of talc, mica, silica, kaolin, polyamide powders, for instance Nylon® powder (Orgasol® from Atochem), poly-β-alanine powder and polyethylene powder, powders of tetrafluoroethylene polymers, for instance Teflon®, lauroyllysine, starch, boron nitride, expanded polymeric hollow microspheres such as those of polyvinylidene chloride/acrylonitrile, for instance Expancel® (Nobel Industrie), acrylic powders, such as Polytrap® (Dow Corning), polymethyl methacrylate particles and silicone resin microbeads (for example Tospearls® from Toshiba), precipitated calcium carbonate, magnesium carbonate, magnesium hydrogen carbonate, hydroxyapatite, hollow silica microspheres (Silica Beads® from Maprecos), glass or ceramic microcapsules, and metal soaps derived from organic carboxylic acids containing from 8 to 22 carbon atoms, e.g., 12 to 18 carbon atoms, for example zinc, magnesium or lithium stearate, zinc laurate and magnesium myristate.

The fillers may be present in an amount ranging from 0.1% to 25%, such as from 1% to 20%, by weight relative to the total weight of the composition.

The composition of the present disclosure may also comprise any additive usually used in cosmetics, such as antioxidants, preserving agents, fragrances, neutralizers, hydrophilic gelling agents, thickeners, vitamins and fibers, and mixtures thereof.

Needless to say, a person skilled in the art will take care to select the optional additional additives and the amount thereof such that the advantageous properties of the composition according to the present disclosure are not, or are not substantially, adversely affected by the envisaged addition.

The hydrophilic gelling agents that may be used in the compositions according to the present disclosure may be chosen from:

homopolymers or copolymers of acrylic or methacrylic acid or the salts and esters thereof, including the products sold under the names Versicol F® or Versicol K® by the company Allied Colloid, Ultrahold 8® by the company Ciba-Geigy, and the polyacrylic acids of Synthalen K type;

copolymers of acrylic acid and of acrylamide sold in the form of the sodium salt thereof under the name Reten® by the company Hercules, sodium polymethacrylate sold under the name Darvan No. 7® by the company Vanderbilt, and the sodium salts of polyhydroxycarboxylic acids sold under the name Hydagen F® by the company Henkel;

polyacrylic acid/alkyl acrylate copolymers of the Pemulen type;

AMPS (polyacrylamidomethylpropanesulfonic acid partially neutralized with ammonia and highly crosslinked) sold by the company Clariant;

AMPS/acrylamide copolymers of the Sepigel® or Simulgel® type, sold by the company SEPPIC, and

polyoxyethylenated AMPS/alkyl methacrylate copolymers (crosslinked or non-crosslinked),

and mixtures thereof.

The water-soluble film-forming polymers mentioned above may also act as water-soluble gelling agents.

The water-soluble gelling polymer may be present in the composition in a solids content ranging from 0.01% to 60% by weight, such as from 0.5% to 40% by weight, and further still for example from 1% to 30% by weight, or even from 5% to 20% by weight, relative to the total weight of the composition.

The term “fiber” should be understood herein as meaning an object of length L and diameter D such that L is very much greater than D, D being the diameter of the circle in which the cross section of the fiber is inscribed. For instance, the ratio L/D (or shape factor) is chosen in the range from 3.5 to 2500, such as from 5 to 500, for example from 5 to 150.

In at least one embodiment, the fibers have a length ranging from 1 μm to 10 mm, such as from 0.1 mm to 5 mm, for example, from 0.3 mm to 3 mm.

The fibers that may be used in the composition of the present disclosure may be chosen from rigid or non-rigid fibers, and may be of synthetic or natural, mineral or organic origin.

As fibers that may be used in the composition according to the present disclosure, mention may be made of non-rigid fibers such as polyamide (Nylon®) fibers or rigid fibers such as polyimideamide fibers, for instance those sold under the names “Kermel” and “Kermel Tech” by the company Rhodia or poly(p-phenyleneterephthalamide) (or aramid) fibers sold under the name Kevlar® by the company DuPont de Nemours.

In at least one embodiment, the composition according to the disclosure is a mascara.

The compositions according to the present disclosure may be prepared according to methods known to those skilled in the art.

The composition according to the present disclosure may be packaged in a container delimiting at least one compartment that comprises the composition, the container being closed by a closing member.

In at least one embodiment, the container is associated with an applicator, for example in the form of a brush comprising an arrangement of bristles maintained by a twisted wire. Such a twisted brush is described, for instance, in U.S. Pat. No. 4,887,622. It may also be in the form of a comb comprising a plurality of application members, which may be obtained by molding. Such combs are described, for example, in French patent FR 2 796 529. The applicator may be solidly attached to the container, as described, for example, in French patent FR 2 761 959. In at least one embodiment, the applicator is solidly attached to a stem, which is itself solidly attached to the closing member.

The closing member may be coupled to the container by screwing. Alternatively, the coupling between the closing member and the container takes place other than by screwing, for instance via a bayonet mechanism, by click-fastening or by tightening. The term “click-fastening” includes any system involving the passing of a rim or bead of material by elastic deformation of a portion, for example by passing the closing member, followed by return to the elastically unstressed position of the said portion after the rim or bead has been passed.

The container may be at least partly made of thermoplastic material. Examples of thermoplastic materials that may be mentioned include polypropylene and polyethylene.

Alternatively, the container is made of a non-thermoplastic material, such as glass or metal (or alloy).

In another embodiment, the container is equipped with a drainer located in the region of the aperture of the container. Such a drainer makes it possible to wipe the applicator and, optionally, the stem to which it may be solidly attached. Such a drainer is described, for example, in French patent FR 2 792 618.

The contents of the patents or patent applications mentioned previously are hereby incorporated by reference.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

The example that follows is presented as a non-limiting illustration of the present disclosure.

The amounts indicated are weight percentages and are expressed relative to the total weight of the composition, unless otherwise indicated.

### EXAMPLES 1 AND 2

### Mascaras

A mascara according to the present disclosure comprising a paraffin wax (Example 2) and a mascara according to the prior art (Example 1) comprising a polar wax (candelilla wax) were prepared.

| | Example 1 | Example 2 |
|---|---|---|
| | (comparative) | (inventive) |
| Hydroxyethylcellulose | 0.9 | 0.9 |
| Triethanolamine | 2.4 | 2.4 |
| Stearic acid | 5.8 | 5.8 |
| Candelilla wax | 30 | |
| Paraffin wax (Cerafine | | 30 |
| 56/58 from Baerlocher) | | |
| Water | qs 100 | qs 100 |

For equal contents, the aprotic wax produced a composition that had a lower consistency (liquid texture) than the composition comprising a polar wax (more consistent creamy texture).

What is claimed is:

1. A composition for coating keratin fibers, comprising, in a cosmetically acceptable continuous aqueous medium, at least one aprotic wax in an amount greater than 25% by weight relative to the total weight of the composition, the composition having a dry extract of greater than or equal to 40%.

2. A composition for coating keratin fibers, comprising, in a cosmetically acceptable continuous aqueous medium,
at least one aprotic wax in an amount greater than or equal to 20% by weight relative to the total weight of the composition and
at least one film-forming polymer chosen from cellulose derivatives.

3. A composition for coating keratin fibers, comprising, in a cosmetically acceptable continuous aqueous medium, less than 5% by weight of protic wax.

4. A composition for coating keratin fibers, comprising a cosmetically acceptable continuous aqueous medium, wherein said composition is free of protic wax.

5. The composition according to claim 1, wherein said at least one aprotic wax is an apolar wax.

6. The composition according to claim 1, wherein the at least one aprotic wax is chosen from: paraffin waxes, microcrystalline waxes, ozokerite, ceresin, synthetic waxes, polyolefin waxes derived from the polymerization of α-olefins of formula R—CH═CH₂ in which R denotes an alkyl radical comprising from 10 to 50 carbon atoms, and Fischer-Tropsch waxes, and mixtures thereof.

7. The composition according to claim 6, wherein said synthetic waxes are chosen from polymethylene wax, polyethylene wax, propylene wax and ethylene/propylene copolymers thereof.

8. The composition according to claim 2, wherein said at least one aprotic wax is present in an amount ranging from 20% to 60% by weight relative to the total weight of the composition.

9. The composition according to claim 1, wherein said at least one aprotic wax is present in an amount ranging from 25% to 60% by weight relative to the total weight of the composition.

10. The composition according to claim 9, wherein said at least one aprotic wax is present in an amount ranging from 27% to 50% by weight relative to the total weight of the composition.

11. The composition according to claim 10, wherein said at least one aprotic wax is present in an amount ranging from 28% to 45% by weight relative to the total weight of the composition.

12. The composition according to claim 1, wherein said aqueous medium is present in an amount ranging from 0.1% to 95% by weight relative to the total weight of the composition.

13. The composition according to claim 12, wherein said aqueous medium is present in an amount ranging from 1% to 80% by weight relative to the total weight of the composition.

14. The composition according to claim 1, further comprising at least one surfactant.

15. The composition according to claim 14, wherein said at least one surfactant is an anionic surfactant chosen from C₁₆-C₃₀ fatty acid salts; polyoxyethylenated fatty acid salts; phosphoric esters and salts thereof; alkyl ether sulfates; sulfosuccinates; isethionates and acylglutamates, and mixtures thereof.

16. The composition according to claim 15, wherein the anionic surfactant comprises at least triethanolamine stearate and/or 2-amino-2-methyl-1,3-propanediol stearate.

17. The composition according to claim 14, wherein said at least one surfactant is present in an amount ranging from 0.1% to 30% by weight relative to the total weight of the composition.

18. The composition according to claim 17, wherein said at least one surfactant is present in an amount ranging from 1% to 15% by weight relative to the total weight of the composition.

19. The composition according to claim 18, wherein said at least one surfactant is present in an amount ranging from 2% to 10% by weight relative to the total weight of the composition.

20. The composition according to claim 1, further comprising at least one film-forming polymer chosen from synthetic polymers, of free-radical type or of polycondensate type, and polymers of natural origin, and mixtures thereof.

21. The composition according to claim 20, wherein said at least one film-forming polymer is chosen from cellulose polymers.

22. The composition according to claim 21, wherein said cellulose polymers are chosen from alkylcelluloses, hydroxyalkylcelluloses, carboxyalkylcelluloses, and mixtures thereof.

23. The composition according to claim 20, wherein said at least one film-forming polymer is present in a solids content ranging from 0.1% to 60% by weight relative to the total weight of the composition.

24. The composition according to claim 23, wherein said at least one film-forming polymer is present in a solids content ranging from 0.5% to 40% by weight relative to the total weight of the composition.

25. The composition according to claim 24, wherein said at least one film-forming polymer is present in a solids content ranging from 1% to 30% by weight relative to the total weight of the composition.

26. The composition according to claim 1, further comprising at least one dyestuff.

27. The composition according to claim 26, wherein said at least one dyestuff is present in an amount ranging from 0.01% to 30% by weight relative to the total weight of the composition.

28. The composition according to claim 1, wherein said composition has a dry extract of greater than or equal to 42% by weight, relative to the total weight of the composition.

29. The composition according to claim 28, wherein said composition has a dry extract of greater than or equal to 45% by weight, relative to the total weight of the composition.

30. The composition according to claim 29, wherein said composition has a dry extract of greater than or equal to 47% by weight, relative to the total weight of the composition.

31. The composition according to claim 30, wherein said composition has a dry extract of up to 70% by weight, relative to the total weight of the composition.

32. The composition according to claim 1, wherein said composition is in the form of a mascara.

33. A process for obtaining a charging makeup on keratin fibers and/or a smooth and uniform deposit on said fibers, said process comprising applying to said keratin fibers a composition comprising, in a cosmetically acceptable continuous aqueous medium, at least one aprotic wax in an amount greater than 25% by weight relative to the total weight of the composition, the composition having a dry extract of greater than or equal to 40%.

34. The process according to claim 33, wherein said keratin fibers are chosen from eyelashes and eyebrows

35. A cosmetic process for caring for or making up keratin fibers, comprising applying to said keratin fibers a composition comprising, in a cosmetically acceptable continuous aqueous medium, at least one aprotic wax in an amount greater than 25% by weight relative to the total weight of the composition, the composition having a dry extract of greater than or equal to 40%.
